(19) United States
(12) Patent Application Publication
(10) Pub. No.: US 2025/0000163 A1
(43) Pub. Date: Jan. 2, 2025

(54) AEROSOL DELIVERY DEVICE WITH MONITORING OF USAGE DATA

(71) Applicant: NICOVENTURES TRADING LIMITED, London (GB)

(72) Inventor: Catalin Mihai BALAN, London (GB)

(21) Appl. No.: 18/708,821

(22) PCT Filed: Oct. 26, 2022
(86) PCT No.: PCT/GB2022/052723

§ 371 Date: May 9, 2024

(30) Foreign Application Priority Data
Nov. 10, 2021 (GB) 2116154.2

(51) Int. Cl. A24F 40/53; A24F 40/10; A24F 40/65

(52) U.S. Cl. CPC A24F 40/53; A24F 40/10; A24F 40/65

(57) ABSTRACT

An aerosol delivery device, the aerosol delivery device comprising a sensor configured to monitor an aspect of usage of the aerosol delivery system by a user; and a controller configured to establish usage data based on monitoring the aspect of usage of the aerosol delivery system by the user over a plurality of puffs comprising between 20 and 150 puffs; wherein the controller is further configured to control an aspect of operation of the aerosol delivery device based on the established usage data.

### FIELD

The present disclosure relates to aerosol delivery systems.

### BACKGROUND

Aerosol delivery systems such as electronic cigarettes (e-cigarettes) generally contain a aerosol generating material, such as a reservoir of a source liquid, which may contain an active substance and/or a flavour, from which an aerosol or vapour is generated for inhalation by a user, for example through heat vaporisation. Thus, an aerosol provision system will typically comprise a aerosol generation chamber containing an aerosol generator, e.g. a heating element, arranged to vaporise or aerosolise a portion of precursor material to generate a vapour or aerosol in the aerosol generation chamber. As a user inhales on the device and electrical power is supplied to the vaporiser, air is drawn into the device through an inlet hole and along an inlet air channel connecting to the aerosol generation chamber where the air mixes with vaporised precursor material to form a condensation aerosol. There is an outlet air channel connecting from the aerosol generation chamber to an outlet in the mouthpiece and the air drawn into the aerosol generation chamber as a user inhales on the mouthpiece continues along the outlet flow path to the mouthpiece outlet, carrying the aerosol with it, for inhalation by the user. Some electronic cigarettes may also include a flavour element in the air flow path through the device to impart additional flavours. Such devices may sometimes be referred to as hybrid devices, and the flavour element may, for example, include a portion of tobacco arranged in the air flow path between the aerosol generation chamber and the mouthpiece such that aerosol/condensation aerosol drawn through the device passes through the portion of tobacco before exiting the mouthpiece for user inhalation.

Aerosol generating systems may be operable to use previous usage data to determine how to control aspects of operation of the device during future operation. In such aerosol generating systems, it is of interest to optimise the storage and use of usage data. Various approaches are described herein which seek to help address or mitigate at least some of the issues discussed above.

### SUMMARY

According to a first aspect of the disclosure, there is provided an aerosol delivery device, the aerosol delivery device comprising: a sensor configured to monitor an aspect of usage of the aerosol delivery system by a user; and a controller configured to establish usage data based on monitoring the aspect of usage of the aerosol delivery system by the user over a plurality of puffs comprising between 20 and 150 puffs; wherein the controller is further configured to control an aspect of operation of the aerosol delivery device based on the established usage data.

According to a second aspect of the disclosure, there is provided a method of controlling an aerosol delivery device, comprising a sensor and a controller, wherein the method comprises controlling the aerosol delivery system to: monitor, using the sensor, an aspect of usage of the aerosol delivery system by a user; establish, using the controller, usage data based on monitoring the aspect of usage of the aerosol delivery system by the user over a plurality of puffs comprising between 20 and 150 puffs; control, using the controller, an aspect of operation of the aerosol delivery device based on the established usage data.

According to a third aspect of the disclosure, there is provided a computer readable storage medium comprising instructions which, when executed by a processor, performs the method according the second aspect.

It will be appreciated that features and aspects of the invention described above in relation to the first and other aspects of the invention are equally applicable to, and may be combined with, embodiments of the invention according to other aspects of the invention as appropriate, and not just in the specific combinations described above.

### BRIEF DESCRIPTION OF THE DRAWINGS

Embodiments of the disclosure will now be described, by way of example only, with reference to the accompanying drawings, in which:

FIG. 1 is a schematic diagram of an aerosol delivery system in accordance with some embodiments of the disclosure.

FIG. 2 is a schematic diagram of a data communication environment, in which an aerosol delivery system is configured to transmit and receive data to and from one or more external electronic devices.

FIG. 3 is a flowchart setting out aspects of operation of an aerosol delivery system according to the present disclosure.

FIG. 4 is a schematic diagram of an example profile, over a plurality of puffs on an aerosol delivery device, of a monitored parameter based on monitoring an aspect of usage of an aerosol delivery device.

FIG. 5 is a schematic diagram of a further example profile, over a plurality of puffs on an aerosol delivery device, of a monitored parameter based on monitoring an aspect of usage of an aerosol delivery device.

### DETAILED DESCRIPTION

Aspects and features of certain examples and embodiments are discussed/described herein. Some aspects and features of certain examples and embodiments may be implemented conventionally and these are not discussed/described in detail in the interests of brevity. It will thus be appreciated that aspects and features of apparatus and methods discussed herein which are not described in detail may be implemented in accordance with any conventional techniques for implementing such aspects and features.

The present disclosure relates to aerosol delivery systems (which may also be referred to as vapour delivery systems) such as nebulisers or e-cigarettes. Throughout the following description the term “e-cigarette” or “electronic cigarette” may sometimes be used, but it will be appreciated this term may be used interchangeably with aerosol delivery system/device and electronic aerosol delivery system/device. Furthermore, and as is common in the technical field, the terms “aerosol” and “vapour”, and related terms such as “vaporise”, “volatilise” and “aerosolise”, may generally be used interchangeably.

Aerosol delivery systems (e-cigarettes) often, though not always, comprise a modular assembly including both a reusable part and a replaceable (disposable) cartridge part. Often the replaceable cartridge part will comprise the aerosol generating material and the vaporiser and the reusable part will comprise the power supply (e.g. rechargeable power source) and control circuitry. It will be appreciated these different parts may comprise further elements depending on functionality. For example, the reusable device part will often comprise a user interface for receiving user input and displaying operating status characteristics, and the replaceable cartridge part in some cases comprises a temperature sensor for helping to control temperature. Cartridges are electrically and mechanically coupled to a control unit for use, for example using a screw thread, bayonet, or magnetic coupling with appropriately arranged electrical contacts. When the aerosol generating material in a cartridge is exhausted, or the user wishes to switch to a different cartridge having a different aerosol generating material, a cartridge may be removed from the control unit and a replacement cartridge attached in its place. Devices conforming to this type of two-part modular configuration may generally be referred to as two-part devices.

It is common for electronic cigarettes to have a generally elongate shape. For the sake of providing a concrete example, certain embodiments of the disclosure described herein will be taken to comprise this kind of generally elongate two-part device employing disposable cartridges. However, it will be appreciated the underlying principles described herein may equally be adopted for different aerosol delivery system configurations, for example single-part devices or modular devices comprising more than two parts, refillable devices and single-use disposable devices, as well as devices conforming to other overall shapes, for example based on so-called box-mod high performance devices that typically have a more boxy shape. More generally, it will be appreciated certain embodiments of the disclosure are based on aerosol delivery systems which are operationally configured to provide functionality in accordance with the principles described herein and the constructional aspects of the aerosol delivery systems configured to provide the functionality in accordance with certain embodiments of the disclosure is not of primary significance.

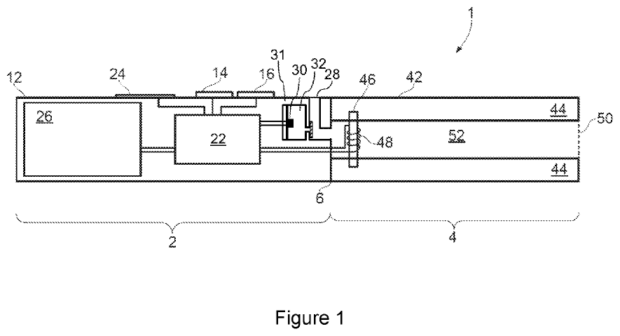

FIG. 1 is a cross-sectional view through an example aerosol delivery system 1 in accordance with certain embodiments of the disclosure. The aerosol delivery system 1 comprises two main components, namely a reusable part 2 and a replaceable/disposable cartridge part 4. The cartridge part 4 is an example of an aerosol generating article or consumable. In normal use the reusable part 2 and the cartridge part 4 are releasably coupled together at an interface 6. When the cartridge part is exhausted or the user simply wishes to switch to a different cartridge part, the cartridge part may be removed from the reusable part and a replacement cartridge part attached to the reusable part in its place. The interface 6 provides a structural, electrical and airflow path connection between the two parts and may be established in accordance with conventional techniques, for example based around a screw thread, magnetic or bayonet fixing with appropriately arranged electrical contacts and openings for establishing the electrical connection and airflow path between the two parts as appropriate. The specific manner by which the cartridge part 4 mechanically mounts to the reusable part 2 is not significant to the principles described herein, but for the sake of a concrete example is assumed here to comprise a magnetic coupling (not represented in FIG. 1). It will also be appreciated the interface 6 in some implementations may not support an electrical and/or airflow path connection between the respective parts. For example, in some implementations an aerosol generator may be provided in the reusable part 2 rather than in the cartridge part 4, or the transfer of electrical power from the reusable part 2 to the cartridge part 4 may be wireless (e.g. based on electromagnetic induction), so that an electrical connection between the reusable part and the cartridge part is not needed. Furthermore, in some implementations the airflow through the electronic cigarette might not go through the reusable part so that an airflow path connection between the reusable part and the cartridge part is not needed. In some instances, a portion of the airflow path may be defined at the interface between portions of reusable part 2 and cartridge part 4 when these are coupled together for use.

The cartridge part 4 may in accordance with certain embodiments of the disclosure be broadly conventional. In FIG. 1, the cartridge part 4 comprises a cartridge housing 42 formed of a plastics material. The cartridge housing 42 supports other components of the cartridge part and provides the mechanical interface 6 with the reusable part 2. The cartridge housing is generally circularly symmetric about a longitudinal axis along which the cartridge part couples to the reusable part 2. In this example the cartridge part has a length of around 4 cm and a diameter of around 1.5 cm. However, it will be appreciated the specific geometry, and more generally the overall shapes and materials used, may be different in different implementations.

Within the cartridge housing 42 is a reservoir 44 that contains aerosol generating material. Aerosol-generating material is a material that is capable of generating aerosol, for example when heated, irradiated or energized in any other way. Aerosol-generating material may, for example, be in the form of a solid, liquid or gel which may or may not contain an active substance and/or flavourants. In some embodiments, the aerosol-generating material may comprise plant material such as tobacco. In some embodiments, the aerosol-generating material may comprise an “amorphous solid”, which may alternatively be referred to as a “monolithic solid” (i.e. non-fibrous). In some embodiments, the amorphous solid may be a dried gel. The amorphous solid is a solid material that may retain some fluid, such as liquid, within it. In some embodiments, the aerosol-generating material may for example comprise from about 50 wt %, 60 wt % or 70 wt % of amorphous solid, to about 90 wt %, 95 wt % or 100 wt % of amorphous solid. The aerosol-generating material may comprise one or more active substances and/or flavours, one or more aerosol-former materials, and optionally one or more other functional material. The aerosol-former material may comprise one or more constituents capable of forming an aerosol. In some embodiments, the aerosol-former material may comprise one or more of glycerine (e.g. vegetable glycerine), glycerol, propylene glycol, diethylene glycol, triethylene glycol, tetraethylene glycol, 1,3-butylene glycol, erythritol, meso-Erythritol, ethyl vanillate, ethyl laurate, a diethyl suberate, triethyl citrate, triacetin, a diacetin mixture, benzyl benzoate, benzyl phenyl acetate, tributyrin, lauryl acetate, lauric acid, myristic acid, and propylene carbonate. The one or more other 30 functional materials may comprise one or more of pH regulators, colouring agents, preservatives, binders, fillers, stabilizers, and/or antioxidants. The aerosol-generating material may be present on or in a support, to form a substrate. The support may, for example, be or comprise paper, card, paperboard, cardboard, reconstituted material, a plastics material, a ceramic material, a composite material, glass, a metal, or a metal alloy.

One or more active constituents (which may herein be referred to as active substances) may comprise one or more physiologically and/or olfactory active constituents which are included in the aerosolisable material in order to achieve a physiological and/or olfactory response in the user. In some embodiments, the active constituent is a physiologically active constituent and may be selected from nicotine, nicotine salts (e.g. nicotine ditartrate/nicotine bitartrate), nicotine-free tobacco substitutes, other alkaloids such as caffeine, cannabinoids, or mixtures thereof. Cannabinoids are a class of natural or synthetic chemical compounds which act on cannabinoid receptors (i.e., CB1 and CB2) in cells that repress neurotransmitter release in the brain. Two of the most important cannabinoids are tetrahydrocannabinol (THC) and cannabidiol (CBD). Cannabinoids may be naturally occurring (Phytocannabinoids) from plants such as cannabis, (endocannabinoids) from animals, and artificially manufactured (Synthetic cannabinoids). Cannabinoids are cyclic molecules exhibiting particular properties such as the ability to easily cross the blood-brain barrier, weak toxicity, and few side effects. Cannabis species express at least 85 different phytocannabinoids, and are divided into subclasses, including cannabigerols, cannabichromenes, cannabidiols, tetrahydrocannabinols, cannabinols and cannabinodiols, and other cannabinoids. such as cannabigerol (CBG), cannabichromene (CBC), cannabidiol (CBD), tetrahydrocannabinol (THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabmolic acid (THCA), and tetrahydrocannabivarinic acid (THCV A).

In the example shown schematically in FIG. 1, a reservoir 44 is provided configured to store a supply of liquid aerosol generating material. In this example, the liquid reservoir 44 has an annular shape with an outer wall defined by the cartridge housing 42 and an inner wall that defines an airflow path 52 through the cartridge part 4. The reservoir 44 is closed at each end with end walls to contain the aerosol generating material. The reservoir 44 may be formed in accordance with conventional techniques, for example it may comprise a plastics material and be integrally moulded with the cartridge housing 42.

The cartridge part further comprises an aerosol generator 48 located towards an end of the reservoir 44 opposite to the mouthpiece outlet 50. An aerosol generator is an apparatus configured to cause aerosol to be generated from the aerosol-generating material. In some embodiments, the aerosol generator is a heater configured to subject the aerosol-generating material to heat energy, so as to release one or more volatiles from the aerosol-generating material to form an aerosol. In some embodiments, the aerosol generator is configured to cause an aerosol to be generated from the aerosol-generating material without heating. For example, the aerosol generator may be configured to subject the aerosol-generating material to one or more of vibration, increased pressure, or electrostatic energy.

It will be appreciated that in a two-part device such as shown in FIG. 1, the aerosol generator may be in either of the reusable part 2 or the cartridge part 4. For example, in some embodiments, the aerosol generator 48 (e.g. a heater) may be comprised in the reusable part 2, and is brought into proximity with a portion of aerosol generating material in the cartridge 4 when the cartridge is engaged with the reusable part 2. In such embodiments, the cartridge may comprise a portion of aerosol generating material, and an aerosol generator 48 comprising a heater is at least partially inserted into or at least partially surrounds the portion of aerosol generating material as the cartridge 4 is engaged with the reusable part 2.

In the example of FIG. 1, a wick 46 in contact with a heater 48 extends transversely across the cartridge airflow path 52 with its ends extending into the reservoir 44 of a liquid aerosol generating material through openings in the inner wall of the reservoir 44. The openings in the inner wall of the reservoir are sized to broadly match the dimensions of the wick 46 to provide a reasonable seal against leakage from the liquid reservoir into the cartridge airflow path without unduly compressing the wick, which may be detrimental to its fluid transfer performance.

The wick 46 and heater 48 are arranged in the cartridge airflow path 52 such that a region of the cartridge airflow path 52 around the wick 46 and heater 48 in effect defines a vaporisation region for the cartridge part 4. Aerosol generating material in the reservoir 44 infiltrates the wick 46 through the ends of the wick extending into the reservoir 44 and is drawn along the wick by surface tension/capillary action (i.e. wicking). The heater 48 in this example comprises an electrically resistive wire coiled around the wick 46. In the example of FIG. 1, the heater 48 comprises a nickel chrome alloy (Cr20Ni80) wire and the wick 46 comprises a glass fibre bundle, but it will be appreciated the specific aerosol generator configuration is not significant to the principles described herein. In use electrical power may be supplied to the heater 48 to vaporise an amount of aerosol generating material (aerosol generating material) drawn to the vicinity of the heater 48 by the wick 46. Vaporised aerosol generating material may then become entrained in air drawn along the cartridge airflow path from the vaporisation region towards the mouthpiece outlet 50 for user inhalation.

As noted above, the rate at which aerosol generating material is vaporised by the vaporiser (heater) 48 will depend on the amount (level) of power supplied to the heater 48. Thus electrical power can be applied to the heater to selectively generate aerosol from the aerosol generating material in the cartridge part 4, and furthermore, the rate of aerosol generation can be changed by changing the amount of power supplied to the heater 48, for example through pulse width and/or frequency modulation techniques.

The reusable part 2 comprises an outer housing 12 having with an opening that defines an air inlet 28 for the e-cigarette, a power source 26 (for example a battery) for providing operating power for the electronic cigarette, control circuitry/controller 22 for controlling and monitoring the operation of the electronic cigarette, a first user input button 14, a second user input button 16, and a visual display 24.

The outer housing 12 may be formed, for example, from a plastics or metallic material and in this example has a circular cross section generally conforming to the shape and size of the cartridge part 4 so as to provide a smooth transition between the two parts at the interface 6. In this example the reusable part has a length of around 8 cm so the overall length of the e-cigarette when the cartridge part and reusable part are coupled together is around 12 cm. However, and as already noted, it will be appreciated that the overall shape and scale of an electronic cigarette implementing an embodiment of the disclosure is not significant to the principles described herein.

The air inlet 28 connects to an airflow path 51 through the reusable part 2. The reusable part airflow path 51 in turn connects to the cartridge airflow path 52 across the interface 6 when the reusable part 2 and cartridge part 4 are connected together. Thus, when a user inhales on the mouthpiece opening 50, air is drawn in through the air inlet 28, along the reusable part airflow path 51, across the interface 6, through the aerosol generation region in the vicinity of the aerosol generator 48 (where vaporised aerosol generating material becomes entrained in the air flow), along the cartridge airflow path 52, and out through the mouthpiece opening 50 for user inhalation.

The power source 26 in this example is rechargeable and may be of a conventional type, for example of the kind normally used in electronic cigarettes and other applications requiring provision of relatively high currents over relatively short periods. The power source 26 may be recharged through a charging connector in the reusable part housing 12, for example a USB connector.

First and second user input buttons 14, 16 may be provided, which in this example are conventional mechanical buttons, for example comprising a spring mounted component which may be pressed by a user to establish an electrical contact. In this regard, the input buttons may be considered input devices for detecting user input and the specific manner in which the buttons are implemented is not significant. The buttons may be assigned to functions such as switching the aerosol delivery system 1 on and off, and adjusting user settings such as a power to be supplied from the power source 26 to an aerosol generator 48. However, the inclusion of user input buttons is optional, and in some embodiments buttons may not be included.

A display 24 may be provided to give a user with a visual indication of various characteristics associated with the aerosol delivery system, for example current power setting information, remaining power source power, and so forth. The display may be implemented in various ways. In this example the display 24 comprises a conventional pixilated LCD screen that may be driven to display the desired information in accordance with conventional techniques. In other implementations the display may comprise one or more discrete indicators, for example LEDs, that are arranged to display the desired information, for example through particular colours and/or flash sequences. More generally, the manner in which the display is provided and information is displayed to a user using the display is not significant to the principles described herein. For example some embodiments may not include a visual display and may include other means for providing a user with information relating to operating characteristics of the aerosol delivery system, for example using audio signalling, or may not include any means for providing a user with information relating to operating characteristics of the aerosol delivery system.

A controller 22 is suitably configured/programmed to control the operation of the aerosol delivery system to provide functionality in accordance with embodiments of the disclosure as described further herein, as well as for providing conventional operating functions of the aerosol delivery system in line with the established techniques for controlling such devices. The controller (processor circuitry) 22 may be considered to logically comprise various sub-units/circuitry elements associated with different aspects of the operation of the aerosol delivery system 1. In this example the controller 22 comprises power supply control circuitry for controlling the supply of power from the power source 26 to the aerosol generator 48 in response to user input, user programming circuitry 20 for establishing configuration settings (e.g. user-defined power settings) in response to user input, as well as other functional units/circuitry associated functionality in accordance with the principles described herein and conventional operating aspects of electronic cigarettes, such as display driving circuitry and user input detection circuitry. It will be appreciated the functionality of the controller 22 can be provided in various different ways, for example using one or more suitably programmed programmable computer(s) and/or one or more suitably configured application-specific integrated circuit(s)/circuitry/chip(s)/chipset(s) configured to provide the desired functionality. The functionality of the controller 22 is described further herein. For example, the controller 22 may comprise an application specific integrated circuit (ASIC) or microcontroller, for controlling the aerosol delivery device. The microcontroller or ASIC may include a CPU or micro-processor. The operations of a CPU and other electronic components are generally controlled at least in part by software programs running on the CPU (or other component). Such software programs may be stored in non-volatile memory, such as ROM, which can be integrated into the microcontroller itself, or provided as a separate component. The CPU may access the ROM to load and execute individual software programs as and when required.

Reusable part 2 comprises an airflow sensor 30 which is electrically connected to the controller 22. In most embodiments, the airflow sensor 30 comprises a so-called “puff sensor”, in that the airflow sensor 30 is used to detect when a user is puffing on the device. In some embodiments, the airflow sensor comprises a switch in an electrical path providing electrical power from the power source 26 to the aerosol generator 48. In such embodiments, the airflow sensor 30 generally comprises a pressure sensor configured to close the switch when subjected to an particular range of pressures, enabling current to flow from the power source 26 to the aerosol generator 48 once the pressure in the vicinity of the airflow sensor 30 drops below a threshold value. The threshold value can be set to a value determined by experimentation to correspond to a characteristic value associated with the initiation of a user puff. In other embodiments, the airflow sensor 30 is connected to the controller 22, and the controller distributes electrical power from the power source 26 to the aerosol generator 48 in dependence of a signal received from the airflow sensor 30 by the controller 22. The specific manner in which the signal output from the airflow sensor 30 (which may comprise a measure of capacitance, resistance or other characteristic of the airflow sensor, made by the controller 22) is used by the controller 22 to control the supply of power from the power source 26 to the aerosol generator 48 can be carried out in accordance with any approach known to the skilled person.

In the example shown in FIG. 1, the airflow sensor 30 is mounted to a printed circuit board 31, but this is not essential. The airflow sensor 30 may comprise any sensor which is configured to determine a characteristic of airflow in an airflow path 51 disposed between air inlet 28 and mouthpiece opening 50, for example a pressure sensor or transducer (for example a membrane or solid-state pressure sensor), a combined temperature and pressure sensor, or a microphone (for example an electret-type microphone), which is sensitive to changes in air pressure, including acoustical signals. The airflow sensor is situated within a sensor cavity 32, which comprises the interior space defined by one or more chamber walls 34. The sensor cavity 32 may also be referred to herein as a sensor chamber 32 (these terms may be used interchangeably), and comprises a region internal to one or more chamber walls 34 in which an airflow sensor 30 can be fully or partially situated. In some embodiments, the airflow sensor 30 is mounted to a printed circuit board (PCB) 31, which comprises one of the chamber walls of a sensor housing comprising the sensor chamber/cavity 32. A deformable membrane is disposed across an opening communicating between the sensor cavity 32 containing the sensor 30, and a portion of the airflow path disposed between air inlet 28 and mouthpiece opening 50. The deformable membrane covers the opening, and is attached to one or more of the chamber walls according to approaches described further herein.

As described further herein, the aerosol delivery device 1 comprises communication circuitry configured to enable a connection to be established with one or more further electronic devices (for example, a storage/charging case, and/or a refill/charging dock) to enable data transfer between the aerosol delivery device 1 and further electronic device(s). In some embodiments, the communication circuitry is integrated into controller 22, and in other embodiments it is implemented separately (comprising, for example, separate application-specific integrated circuit(s)/circuitry/chip(s)/chipset(s)). For example, the communication circuitry may comprise a separate module to the controller 22 which, while connected to controller 22, provides dedicated data transfer functionality for the aerosol delivery device. In some embodiments, the communication circuitry is configured to support communication between the aerosol delivery device 1 and one or more further electronic devices over a wireless interface. The communication circuitry may be configured to support wireless communications between the aerosol delivery device 1 and other electronic devices such as a case, a dock, a computing device such as a smartphone or PC, a base station supporting cellular communications, a relay node providing an onward connection to a base station, a wearable device, or any other portable or fixed device which supports wireless communications. Wireless communications between the aerosol delivery device 1 and a further electronic device may be configured according to known data transfer protocols such as Bluetooth, ZigBee, WiFi, Wifi Direct, GSM, 2G, 3G, 4G, 5G, LTE, NFC, RFID. More generally, it will be appreciated that any wireless network protocol can in principle be used to support wireless communication between the aerosol delivery device 1 and further electronic devices. In some embodiments, the communication circuitry is configured to support communication between the aerosol delivery device 1 and one or more further electronic devices over a wired interface. This may be instead of or in addition to the configuration for wireless communications set out above. The communication circuitry may comprise any suitable interface for wired data connection, such as USB-C, micro-USB or Thunderbolt interfaces. More generally, it will be appreciated the communication circuitry may comprise any wired communication interface which enables the transfer of data, according to, for example, a packet data transfer protocol, and may comprise pin or contact pad arrangements configured to engage cooperating pins or contact pads on a dock, case, cable, or other external device which can be connected to the aerosol delivery device 1.

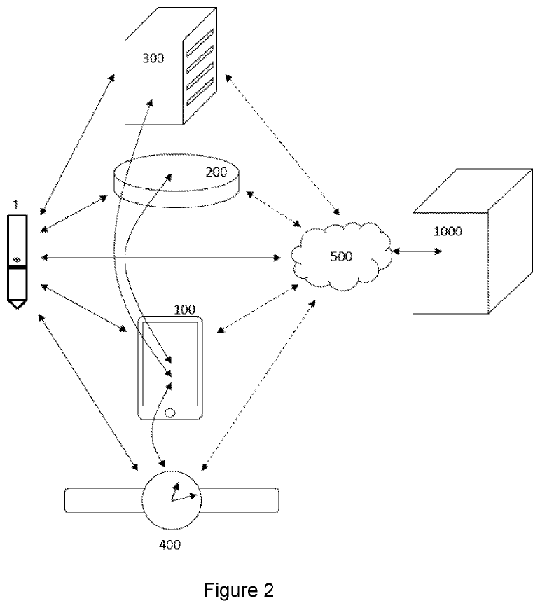

Referring now to FIG. 2, the aerosol delivery device 1 (or more generally any delivery device as described elsewhere herein) may operate within a wider delivery system/aerosol delivery system comprising one or more of a smartphone 100, a dock 200 (e.g. a storage/charging case or home refill and/or charging station), a personal computer (PC) 300, or a wearable device 400 (e.g. a smart watch), and a server 1000 (where communications with the server may be supported over an internet or other packet data connection 500). The electronic devices comprised in the system may communicate with the aerosol delivery device 1, either directly (as shown with solid arrows) or indirectly (shown with dashed arrows). This delivery system/aerosol delivery system may be referred to as a delivery ecosystem/aerosol delivery ecosystem.

An example aerosol delivery device 1 such as an e-cigarette may communicate directly with one or more other classes of device in a wider aerosol delivery ecosystem, including but not limited to a smartphone 100, a dock 200 (e.g. a recharging case or home refill and/or charging station), a personal computer (PC) 300, or a wearable device 400 (e.g. a smart watch). In a similar manner, the aerosol delivery device 10 such as an e-cigarette may communicate directly with another device of the same class, i.e. another aerosol delivery device. As noted above, these devices may cooperate in any suitable configuration to form a delivery system 1. This communication may be supported by wired communication circuitry of the aerosol delivery device 1 (for example, using an interface such as USB-C, micro-USB, Thunderbolt, or another wired communication interface as described further herein), or by wireless communication circuitry of the aerosol delivery device 1 (for example, a Bluetooth, ZigBee, WiFi, Wifi Direct, GSM, 2G, 3G, 4G, 5G, LTE, NFC or RFID module, or another wireless communication interface as described further herein). The aerosol delivery device 1 may be configured to connect to different ones of other classes of device using different wired or wireless communication protocols, and a data connection between the aerosol delivery device 10 and any given second device may be established using wired and/or wireless communication. It will be appreciated that other classes of device comprised in the wider delivery system may comprise communication circuitry for wired or wireless data transmission similar to that set out further herein in relation to the aerosol delivery device 1. Accordingly, a smartphone 100, a dock 200 (e.g. a home refill and/or charging station), a personal computer (PC) 300, a wearable device 400 (e.g. a smart watch) or a server 1000 may be equipped with communication circuitry comprising a Bluetooth, ZigBee, WiFi, Wifi Direct, GSM, 2G, 3G, 4G, 5G, LTE, NFC, RFID or other wireless transmission module, and/or a wired interface such as USB-C, micro-USB, Thunderbolt or other wired interface.

Communication circuitry of the aerosol delivery device 1 (implemented as a single module or separate modules) may enable it to communicate with different ones of the further classes of device using different wired and/or wireless data transmission protocols.

The aerosol delivery device 1 and other classes of device in the delivery system may communicate directly or indirectly with a server 1000 via a network such as the internet 500 or other suitable packet data protocol known to the skilled person (such as those set out in the 3GPP standards for cellular wireless communications). The aerosol delivery device 1 may establish such communication directly, using one of the wireless communication protocols described further herein to communicate with communication node/transceiver infrastructure (such as a ‘base station’ or ‘evolved node-B’ in LTE terminology) which provides connectivity with the server 1000 (e.g. over a backhaul communication link). Alternatively or in addition, the aerosol delivery device 1 may establish communication with the server 1000 via another device in the delivery system 1, for example using a wired or wireless communication protocol to communicate with a smartphone 100, a dock/case 200, a personal computer (PC) 300, or a wearable device 400 which then communicates with the server 1000 (for example, via the internet 500) to either relay data to or from the aerosol delivery device 1, report upon its communications with the aerosol delivery device 1, or exchange information inferred about the aerosol delivery device 1 without a connection to the aerosol delivery device 1 being established. The smartphone 100, dock 200, or other device within the delivery ecosystem, such as a point of sale system/personal computer (PC) 300, may optionally act as a hub for one or more aerosol delivery devices 1 that only have short range transmission capabilities (provided, for example, by communication circuitry comprising a Bluetooth or RFID module). Such a hub may thus extend the battery life of an aerosol delivery device 1 whilst enabling data to be exchanged between the aerosol delivery device 1 and further devices of the aerosol delivery system 1 (for example, server 1000).

The other classes of device in the aerosol delivery system 1, such as the smartphone 100, dock 200, personal computer (or, for example, a point of sale system) 300 and/or wearable 400 may also communicate indirectly with the server 1000 via a relay device, either to fulfil an aspect of their own functionality, or on behalf of the aerosol delivery system 1 (for example as a relay or co-processing unit). These devices may also transfer data with each other, either directly or indirectly via any of the wired or wireless communication protocols set out further herein. Thus, in embodiments of the disclosure, a given first and second device of the delivery system may generally be in either a connected or unconnected state. The unconnected state may also be referred to as an idle state, and in such a state a given first device may not be detectable by other second devices (i.e. the first device is not transmitting any signalling enabling its existence and/or identity to be determined), or it may be available for establishing a connection with a second device (i.e. it may be advertising its existence/identity using beacon/advertisement signalling). In a connected state, the first and second devices are configured such that data may be transferred from the first to the second device (e.g. ‘uplink’ transmission) and/or transferred from the second to the first device (e.g. ‘downlink’ transmission). Accordingly, establishment of a connection between a first and second device may be considered to comprise the establishment of any state wherein the two devices can exchange data, regardless of the direction of data transfer. Non-limiting examples of connected states are the establishment of an RRC connected state according to the Long Term Evolution (LTE) standard, or a bonded/paired state according to the Bluetooth standard. When a first and second device of the delivery system are configured to communicate wirelessly, a transition from an unconnected to a connected state will generally follow a procedure such as the following. In an initial enquiry step, a first device (for example, an aerosol delivery device 1) establishes the existence of a second device (for example, a smartphone 100 or case 200) by receiving a beacon signal or other identifying signal/message from the second device. In an authentication step, the first and second devices exchange messaging to establish information relating to the data transfer protocol to be used for exchanging data (for example comprising coding and encryption parameters to be used when exchanging data packets). In a data transfer step, the first and second devices transfer data over an air interface established in accordance with an agreed data transfer protocol (for example, Bluetooth, ZigBee, RFID, or other protocols described further herein). This data transmission may be bi- or uni-directional. The data communication process for wired communications may be broadly similar with the difference that data is transmitted over a wired interface as opposed to a wireless interface. Further aspects of implementation for establishment of wireless and wired communications may be found in the standard documents for communication protocols such as those listed further herein.

It will be appreciated that references herein to functionality of a controller 22 of an aerosol delivery device, in terms of monitoring usage of the aerosol delivery device, storing usage data, and establishing parameters for controlling operation of the aerosol delivery device on the basis of such usage data, may be ‘offloaded’ to a processor or controller associated with an external electronic device having a wired or wireless data connection to the aerosol delivery device, as shown schematically in FIG. 2, and described in the accompanying text. In the case where some or all of the computer operations occur on an external electronic device, resulting parameters determined for control of the aerosol delivery device may, as appropriate, are transmitted back to the aerosol delivery device over a wired or wireless data connection. Thus, throughout the present disclosure, it will be appreciated that when operations are referred to in which (i) user inputs such as selection of parameters and device modes are provided to a user input interface of the reusable device part 2. (ii) information is displayed to a user on a display of the reusable device part 2, and (iii) information is processed by a controller 22 (e.g. for determining how to control at least one aspect of the operation of the aerosol delivery system on the basis of a target operating characteristic of the aerosol delivery system and identifying information for a cartridge/consumable part), one or more of these functions may be carried out by an external electronic device (e.g. a smartphone, a refill/charging dock or case, a personal computer (PC), a wearable device (e.g. a smart watch) or a server) to which the aerosol provision system is configured to connect via a wired or wireless communication protocol using a wired or wireless data interface between the aerosol delivery system 1 and the external electronic device.

The controller 22 of the aerosol delivery device is configured to monitor at least one aspect of usage of the aerosol delivery device by a user. For example, inhalation events (also referred to herein as ‘puffs’) may be detected on the basis of signals received by controller from an activation device comprising an airflow sensor 30. When a rate of airflow (and/or a drop in pressure) detected by the airflow sensor 30 increases beyond a predetermined threshold, a puff may be determined by the controller 22 to have started, and the time of the puff initiation may be stored in a memory element associated with controller 22. When the rate of airflow (and/or a drop in pressure) detected by the airflow sensor 30 drops again below the predetermined threshold (or a second, different threshold to account for sensor hysteresis), the puff may be determined by the controller 22 to have ended, and the time of the puff end may also be stored in a memory element associated with controller 22. This timing information may be used to determine the duration of each of a plurality of puffs, and the delay between subsequent puffs. A supply of power to the heater 48 may be initiated and ended by the controller at the puff start and puff end times.

As mentioned above, a button 14, which represents a form of manual activation device, may be located on the outer housing of the body 20. The button 14 may be implemented using any appropriate mechanism which is operable to be manually activated by the user—for example, as a mechanical button or switch, a capacitive or resistive touch sensor, and so on. The puff start and end times described above may in other embodiments be determined based on the times at which a user respectively presses and releases a button 14, with a supply of power to the heater 48 being initiated and ended at these same points in time.

Where the aerosol delivery device 1 comprises an airflow sensor 30, the controller 22 may monitor a strength of user inhalation associated with each of a plurality of puffs and output by the airflow sensor 30 and store this in the memory element. This may comprise for example a maximum inhalation strength during each puff, or a time-varying profile of inhalation strength during each puff. The power supplied to the heater during each puff may also be monitored and stored in the memory element. The power supplied to the heater by the controller 22 may be adjusted by a user from puff to puff or from session to session using a user input interface associated with the aerosol delivery device, or associated with an external electronic device, such as for example a smartphone 100 having a data connection to the aerosol delivery device. For example, a power level to be supplied to the heater may be selected via a software application (APP) running on a smartphone 100, which this power level being indicated to the controller 22 via a wireless data connection between smartphone 100 and the aerosol delivery device as described further herein.

In the present disclosure, the term ‘inhalation session’ may be used to refer to a sequence of user inhalations (i.e. puffs). The sequence may correspond to a characteristic pattern, such as a high-to-low volume sequence of puffs, where the volume of aerosol delivered per puff generally decreases over subsequent puffs. Alternatively, an inhalation session may correspond to a predetermined number, or range, of inhalations on the aerosol delivery device by the user. For example, a session may be defined as 10 inhalations or between 8 to 12 inhalations. Additionally, or alternatively, a session may be defined by a predefined time from an initial inhalation on the aerosol delivery device by the user. For example, the predetermined time may be four minutes. Hence, a session can be defined when the total number of inhalations reaches 8 to 12 inhalations and/or when the time elapsed from the first inhalation reaches four minutes. It should be appreciated that the values for the predetermined number of inhalations and predetermined time have been given purely as an example, and other numbers and times may be used in other implementations as appropriate.

Alternatively or in addition, in an embodiment of the present disclosure an inhalation session may be separated from another session by period of non-use, with a first session being considered to have finished and second subsequent session to have started when a threshold duration between the last puff of the first session and the first puff of the second session is longer than a threshold duration. For example, the threshold duration may be selected for ease of comprehension by the user (e.g. 5, 10, 15, 20, 25, 30, 35, 40, 45, 50 or 55 minutes, or more preferably a period in the range 5 to 45 minutes, or more preferably a period in the range 10 to 30 minutes, or still more preferably a period in the range 15 to 20 minutes), or it may be selected on a pharmokinetic basis, such as the so-called half-life of an active substance comprised in the aerosol generating material which is aerosolised by the aerosol delivery device. Hence an inhalation session may comprise a characteristic pattern and/or may be separated from another session by a threshold duration of non-use, and usage data may comprise information about a number of puffs in each of a plurality of sessions, and the duration of each of a plurality of sessions, spanning a period over which N puffs are taken on the device.

In embodiments of the present disclosure, usage data/usage information may comprise parameters derived based on sensing other interactions of a user with the aerosol delivery system which are not directly related to inhalation, but which are nonetheless recorded with respect to a timeline of puffs on the device. For example, usage data may comprise:
Data about location over time of the aerosol delivery device, or an external electronic device such as a smartphone, based on GPS location tracking over time, according to GPS tracking approaches known to the person skilled in the art, over a time period spanning a plurality of N puffs.
Data relating to selection of parameters or manual input of data by a user to the aerosol delivery device or an associated electronic device of the aerosol delivery system. For example usage data may comprise a record of how a user has adjusted a level of power to be supplied to the heater 48 of the aerosol delivery device over a time period spanning a plurality of N puffs.
Data relating to physical interactions with the user with the aerosol delivery device which are not directly related to airflow through the device. For example, suitable sensors may be positioned on an outer housing of the aerosol delivery device according to approaches known to the skilled person to monitor a temperature of the user's skin as they hold the device, or the acceleration of the device, to give a few non-limiting examples, over a time period spanning a plurality of N puffs.

The inventors have recognised that it may be desirable to optimise the amount of usage data retained by a controller of an aerosol delivery device, in view of the overheads involved in storing and processing such data. Particularly in circumstances where the controller 22 is configured to monitor a plurality of aspects of usage of the aerosol delivery device, such that a plurality of types of usage data are established (for example, relating to puff characteristics, session characteristics, device location characteristics, and user input characteristics), this monitoring may result in a significant rate of data generation, which requires storage on a memory element of the controller 22. In view of seeking to simplify the controller 22 to reduce cost and/or the power draw on the battery/power supply 26, it may be desirable to optimise the amount of data to be stored to find a compromise between storing sufficient raw monitoring data to enable establishment of meaningful usage data which is representative of a user's characteristic behaviour, and the amount of storage space required to store this data. Moreover, even if there is not a technical requirement to limit the storage space available for storing usage data, it may be desirable to optimise an amount of usage data taken into account when establishing how to control an aspect of operation of an aerosol delivery system based on established usage data. The inventors have recognised that by optimising the amount of usage data used to establish how to control aspects of operating of an aerosol delivery system, processing overheads for a controller of the aerosol delivery system can be minimised.

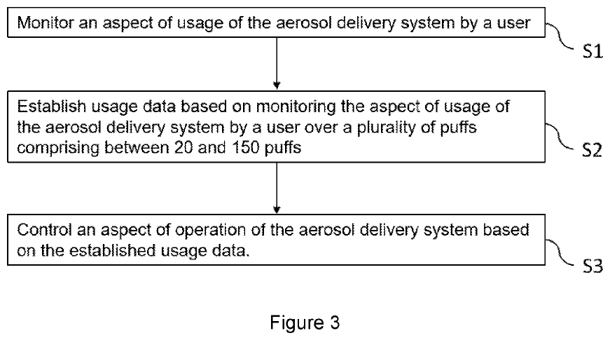

FIG. 3 shows a flowchart of an approach according to the present disclosure, according to which, in a first step S1, an aspect of usage of the aerosol delivery system by a user is monitored by using a sensor. In a second step S2, usage data is established, based on monitoring the aspect of usage of the aerosol delivery system by the user over a plurality of puffs comprising between 20 and 150 puffs. In a third step S3, an aspect of operation of the aerosol delivery system is controlled by a controller based on the established usage data.

In embodiments of the present disclosure, usage data is established based on monitoring one or more aspects of usage over a time period spanning a number of puffs N comprising between 20 and 150 puffs. In some embodiments, the actual number of puffs N may be selected to be characteristic of a user's patterns of usage of the aerosol delivery device. It has been recognised that a user's usage of the device may follow a generally cyclical pattern with a certain periodicity. For example, the user may generally use the aerosol delivery device over sessions of puffs which have similar characteristics in terms of characteristics such as length of session, average inhalation strength of puffs in the session, average length of puffs in the session, and other parameters. Other aspects of usage of the aerosol delivery device may be similar across each of a first and second time period of equal length, where the length of the time period covers a characteristic number of puffs N. Thus aspects of usage such as a pattern of location of the device, a pattern of user inputs on the device (such as variation of heater power settings) may generally be similar in both of a first and second time period, each period covering approximately a characteristic number of puffs N. For example, where the time period covering the N puffs equates to approximately 24 hours, aspects of usage of the aerosol delivery device by the user for successive periods may be assumed to be similar based on the assumption that a user may generally follow a similar routine and experience a similar pattern of interaction with the aerosol delivery device over the course of each day.

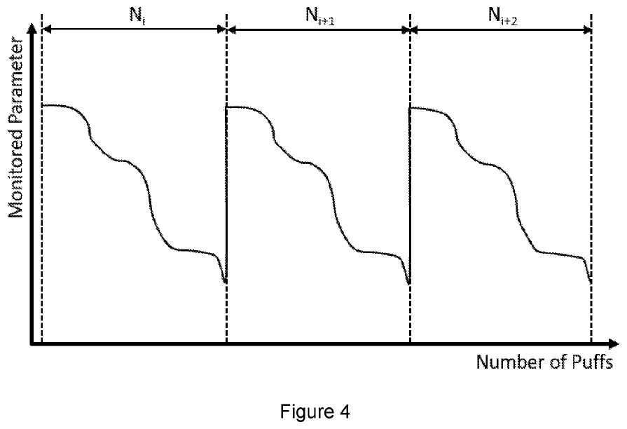

Thus in embodiments of the present disclosure, a number of puffs N of between 20 and 150 puffs is established, over which usage data is established based on monitoring usage of the aerosol delivery system. A suitable value for N within the range of between 20 and 150 puffs may be established in one of a number of different ways as further described herein. FIG. 4 shows schematically an example profile over number of puffs on an aerosol delivery device of a monitored parameter based on monitoring an aspect of usage of an aerosol delivery device. The monitored parameter may relate to any aspect of usage described herein, and be dependent on the individual context in which the approach is implemented.

The specific parameter is not considered of particular significance, and will differ in different embodiments. Thus in one non-limiting example, the monitored parameter is an average puff duration. FIG. 4 shows schematically that the magnitude of the average puff duration (which may be a windowed average over a plurality of puffs) follows a cyclic pattern, such that over a first plurality of puffs Ni, the average puff duration begins higher and gradually drops over the course of the Ni puffs. As the user moves into the next set of puffs (set Nᵢ₊₁), the average puff duration moves back to a higher value, and again drops off again over the Nᵢ₊₁ puffs. This cycle repeats again over the subsequent cycle of Nᵢ₊₂ puffs. Though FIG. 4 shows the monitored parameter profile as the same over each of sets of puffs Ni, Nᵢ₊₁, and Nᵢ₊₂, and the pluralities of puffs Ni, Nᵢ₊₁, and Nᵢ₊₂ comprising the same number of puffs, there may be some variation between the number of puffs in each of Ni, Nᵢ₊₁, and Nᵢ₊₂, and between the profiles of the monitored parameter (e.g. puff duration). What is significant is that the profile of the monitored parameter follows a generally cyclic nature with a generally characteristic periodicity in terms of number of puffs N in each cycle. Thus in embodiments of the disclosure, usage data established on the basis of monitoring an aspect of usage (via a monitored parameter) over a plurality of puffs N may be assumed to be representative of usage of the device in other periods (e.g. in the example of FIG. 4, the usage over the plurality of puffs Nᵢ is representative of the usage over pluralities of puffs Nᵢ₊₁, and Nᵢ₊₂).

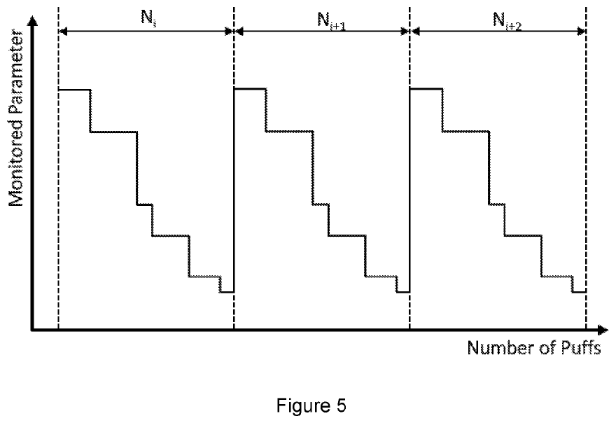

FIG. 5 is similar to FIG. 4, except that in this instance, the monitored parameter comprises a power level selected by the user (or automatically selected by controller 22). Thus over a first plurality of puffs Ni, a relatively high power is initially provided to the heater over a first subset of puffs, following which the power level is gradually reduced over subsequent subsets of puffs in the plurality of puffs Nᵢ. A similar profile of power level is observed in subsequent pluralities of puffs Nᵢ₊₁, and Nᵢ₊₂. Thus N puffs may be taken to be a number of puffs which is representative of the periodicity over which the level of power provided to the heater varies (e.g. by input from a user, or automatically by controller 22). Though FIGS. 4 and 5 refer to a monitored puff duration, and power delivered to a heater, respectively, it will be appreciated any monitored parameter/usage parameter described herein may be substituted in other embodiments.

Where determination of N is based on determining a characteristic period of cyclic change in an aspect of usage of the aerosol delivery device by the user, this determination may be carried out by a software routine running on controller 22, or on a processor associated with an external electronic device as shown in FIG. 2. This processing may use data representing the value of a monitored parameter over a plurality of puffs (e.g. taken over a period of hours, days, or weeks), to determine an appropriate value for N using an appropriate algorithm/statistical technique. For example approaches utilising Fourier-based spectral decomposition of the monitored parameter data may be used to establish the characteristic periodicity of the monitored parameter with respect to number of puffs. However, any statistical approach for determining characteristic periodicity in a time-varying signal may be used.

In other embodiments, the number of puffs in the plurality of puffs N may comprise a number of puffs over which an average of a monitored parameter is determined to be representative of the monitored parameter. For example, due to the degree of noise in the value of the monitored parameter over a plurality of puffs, a number of puffs of N>i may be taken into account when averaging the parameter over a plurality puffs to avoid skewing of an average such as the mean or median of the monitored parameter, where i may be determined using statistical approaches known in the art, for establishing an appropriate sample size to use. A suitable value for N for a given aspect of usage may be determined experimentally or by modelling, and may be based on experimental data derived by monitoring an aspect of usage using approaches set out herein over time, for one or more users. Standard statistical approaches for selecting appropriate sample sizes, known to the skilled person, may be used to select an appropriate value for i.

An aerosol delivery device according to the present disclosure may in some instances ‘learn’ an appropriate value for N based on monitoring an aspect of usage of the device during an initial training period as the user interacts with the device. A software routine running on controller 22, or on a processor associated with an external electronic device as shown in FIG. 2, may use data representing the value of a monitored parameter over a plurality of puffs (e.g. taken over a period of hours, days, or weeks), to determine an appropriate value for N, according to any of the approaches set out further herein by which an appropriate value for N is determined based on analysis of usage data. In some instances a value of N may be based on monitoring an aspect of usage of a plurality of aerosol delivery devices by a plurality of users with, with the monitored parameter data derived during this monitoring being collected by, for example, a server 1000 via wireless data connections between the server 1000 and each of the plurality of aerosol delivery devices, using data connection approaches described further herein.

In other embodiments, the value of N may be determined by monitoring the number of puffs over a plurality of respective 24 hour periods, and setting N as an average number of puffs per 24 hour period. The inventors have recognised that some values of N provide usage data which is particularly representative of aspects of usage of an aerosol delivery device by a user. For example, the value of N is preferably between 10 and 300 puffs, or between 20 and 250 puffs, or between 30 and 200 puffs, or between 40 and 175 puffs, or between 50 and 150 puffs, or between 60 and 140 puffs, or between 70 and 130 puffs, or between 80 and 120 puffs, or between 90 and 110 puffs, or between 95 and 105 puffs. In some embodiments, the value of N may be between 20 and 150 puffs, between 20 and 100 puffs, or between 20 and 50 puffs, the latter of which may be particularly appropriate when the aspect of operation of the aerosol delivery system is controlled in a given day based on usage data established earlier in the same day according to any of the approaches set out further herein.

Once a value has been determined for N, this value may be used to control aspects of the operation of the aerosol delivery device, according to one or more of the following approaches. It will be appreciated that the value of N (i.e. the number of puffs in the plurality of puffs over which usage data is established based on monitoring the aspect of usage of the aerosol delivery system by the user) is generally predefined by the controller 22 of the aerosol delivery system. By ‘predefined’ it is meant that the number of puffs over which usage data is to be established and used to control operation of the aerosol delivery system is determined by the controller 22 in advance of the controller obtaining the usage data and controlling the operation of the aerosol delivery system on the basis of the usage data.

In some embodiments, the value of N is used to determine how much monitoring data obtained via monitoring an aspect of usage of an aerosol delivery device to store in a memory element of the aerosol provision device. The memory element may be integrated in controller 22, or may be a standalone memory element (for example, a micro-SD card or similar flash memory).

For example, a hardware or virtual buffer comprised in a memory element associated with controller 22 may be configured to provide N data slots, each of which can store a value of a monitored parameter associated with an individual puff, on the basis of which usage data is established according to approaches set out further herein. When a puff is taken, the value of the monitored parameter associated with the puff may be written to the lead slot of the buffer, with the remaining N−1 values shifted one slot along the buffer (such that the value relating to the least recent puff is no longer contained in the buffer). The usage data may then be re-established based on the updated values in the buffer. For example, where the values stored in the buffer comprise puff duration values, and the usage data used to control an aspect of operation of the aerosol delivery system comprises a mean of the last N puff duration values, the mean may be recomputed following each puff. This approach may be considered a windowed-averaging approach to generation of usage data based on monitoring an aspect of usage of the aerosol delivery device, where the usage data is a windowed average over N puffs of a monitored parameter derived from monitoring an aspect of usage of the aerosol delivery device by the user. It will be appreciated this approach may be applied in respect of any usage data which is associated with each puff, as described further herein. In aerosol delivery devices where the value of N can be updated over time (for example, where N is recalculated on an hourly, daily, or weekly basis, based on analysing variation of a monitored parameter over time as described further herein), the size of the buffer may be dynamically changed. This may be advantageous as it can allow storage capacity of a memory element comprising the buffer to be optimised, so that space not allocated to the buffer can be assigned by the controller for other purposes.

According to embodiments of the current disclosure, the controller 22 the aerosol delivery system and/or a processor of an external electronic device such as described further herein, are configured to determine one or more control parameters for controlling an aspect of operation of the aerosol delivery device, based on usage data derived by monitoring one or more aspects of usage of the aerosol delivery device by the user over a period of time corresponding to N inhalation events or ‘puffs’ by a user on the aerosol delivery device (which may be distributed amongst M sessions).

Aspects of device operation may be determined by one or more device control parameters, which may comprise: parameters related to control of a heater or other aerosol generating element; parameters related to control of visual, audible and/or haptic indications provided to a user; parameters related to data connectivity, such as upload of usage data from the aerosol delivery device to one or more external devices in a wider aerosol delivery system; or any other parameters for control of aspects of operation of an aerosol delivery device known to the skilled person. Device parameters are generally used as inputs to controller 22 to cause operating parameters of the aerosol delivery device to be changed, or may be used to control aspects of operation of one or more external electronic devices such as shown in FIG. 2 and described in the accompanying description. For example, a device control parameter comprising a heater control parameter may specify a power level to be supplied to the heater from battery/power source 26 to heater 48 by controller 22; a device control parameter comprising an indication control parameter may be used controller 22 to control the provision of a visual, aural, or haptic notification or prompt to a user; a device control parameter comprising a cessation control parameter may be used by controller 22 to control periods of time during which a user respectively is and is not able to use the aerosol delivery device to generate aerosol. More generally, a device control parameter may comprise any parameter usable by a controller 22 of an aerosol delivery system to modify some aspect of operation of the aerosol delivery system, and may be based on aspects of operation of aerosol delivery devices known to the skilled person.

In some embodiments of the present disclosure, the monitored aspect of usage comprises one or more properties of a plurality of user puffs, comprising N puffs, and the controlled aspect of operation of the aerosol delivery device is an amount of power to be provided to a heater of the device in a subsequent puff or session. The properties of the N puffs may be considered indicative of a demand for aerosol by the user, such that the usage data may be considered to be representative a pattern of such demand. Thus, the usage data established over a plurality of N puffs may be used to control the manner in which aerosol is generated in one or more subsequent puffs.

In an embodiment, usage data for the preceding N puffs is used to control a heating duration and/or heater power level in a subsequent puff. For example, after a puff, the average mass loss of aerosol per puff may be estimated based on computing a mass loss of aerosol in each of the preceding N puffs, where N is between 20 and 100 puffs. The mass loss of aerosol may be estimated using a function of the heater power and the puff duration, and optionally the airflow rate as determined based on the output of an airflow sensor. These parameters may be input into a fitted model, derived by experimentation to quantify mass loss of aerosol over different puff lengths, heater power levels, and optionally, airflow rates. Thus once the average mass loss of aerosol per puff is estimated based on the preceding N puffs, the heating duration and/or heater power level may be controlled in a subsequent puff to target the same mass loss value. This may be based on assuming the puff length and optionally airflow rate in a subsequent puff will be the average of the preceding N puffs, and then controlling the power to the heater, based on the fitted model, to target the mass loss value. In other embodiments, the control parameter may not be directly related to control of heating, but may be related to, for example, provision of user indications. For example, based on the average mass loss of aerosol in each of the previous N puffs, and information about the amount of aerosol generating material remaining in a consumable part coupled to the aerosol delivery system, the controller may estimate a remaining number of available puffs using an assumption that the future puffs will be associated with the average mass loss per puff. This estimated puff number may be displayed to a user on a user interface of the aerosol delivery device, or on an APP running on a smartphone with a data connection to the aerosol delivery system.

Where the controller is configured to estimate user demand for a next puff or inhalation session by the user, this may be determined by controller 22 based on any suitable property, or combination of properties, of the plurality of preceding puffs, comprising N puffs, as represented, for example, by usage data comprising information about puff start and end times, puff strength, or power delivered to the heater per puff, as determined by controller 22 based on monitored parameters of aspects of usage of the aerosol delivery device as described further herein.

For example, with reference to FIG. 4, usage data may be derived which indicates that over N puffs, the demand for aerosol generally decreases, based on identifying that over N puffs, an average puff duration of puffs taken by the user follows a decreasing pattern.

In some instances, the value of a control parameter may be modified over a sequence of Nᵢ₊₁ puffs based on usage data comprising a profile of a monitored parameter over a preceding sequence of Ni puffs, where the sequences Nᵢ₊₁ and Ni may or may not overlap. Thus for example, where a monitored parameter is puff duration, as in FIG. 4, the power delivered to the heater 48 by controller 22 during the nth puff of a series of Nᵢ₊₁ puffs may be based on an average duration of puffs in a preceding N puffs (i.e. the power for the nth puff, Pₙ, may be calculated as Pₙ=f(t̅), where t̅ is the average puff duration during the preceding N puffs). This scheme recognises that puff duration may be indicative of user demand for aerosol, such that longer puffs indicate a greater demand for aerosol. Accordingly, scaling up the amount of power in subsequent puffs based on the average duration of a preceding N puffs, or otherwise providing more aerosol to the user per unit puff time, may provide a more satisfying experience for the user. Alternatively, the power Pₙ delivered during the nth puff of a series of Nᵢ₊₁ puffs may be calculated as Pₙ=f(tₙ), where tₙ is the duration of the nth puff in the preceding series of N puffs (e.g. series Ni), or an average duration of a plurality of puffs centred on the nth puff in the preceding series of N puffs. This latter scheme recognises that where N puffs is a number of puffs representative of the periodicity of a cycle of usage of the device (e.g. in terms of demand for aerosol), the user demand at a particular position in a previous cycle (e.g. a previous N puffs) may be indicative of user demand at the same position in a current cycle (e.g. a current N puffs). Though these examples have been described in terms of a monitored parameter comprising puff duration, and a control parameter comprising power provided to a heater, it will be appreciated any suitable monitored parameter or control parameter described herein can be substituted in these examples.

In embodiments of the disclosure, the control parameter may be a cessation control parameter. The value of a cessation control parameter for one or more puffs may be established based on usage data comprising a characteristic rate of aerosol generation, based on monitoring puff duration and timing over a preceding N puffs. For example, usage data comprising an amount of aerosol per puff may be established, based on a function such as f(t,P), where t is the puff duration of puffs in the plurality of puffs N, and P is the power delivered to the heater for each of the plurality of puffs N. Based on estimating the amount of aerosol delivered in each of the puffs in a preceding plurality of N puffs, the controller may establish cessation control parameters used to prevent usage of the aerosol delivery device by a user to generate aerosol for a certain period of time. For example, a cessation control scheme may comprise preventing a user from generating aerosol for a predefined period of time when a rate of aerosol generation in a second plurality of puffs is determined to exceed a rate of aerosol generation in a previous plurality of N puffs. For example, a rate of aerosol generation over the last N puffs may be calculated as a mass of aerosol per unit time, by estimating, at the controller, a mass loss of aerosol per one of the N puffs, based for example on the power supplied to the heater and duration of each puff (i.e. using a predefined function linking heater power to a rate of aerosol generation, established via modelling and/or experimentation), and calculating a mass loss of aerosol over the elapsed time in which the N puffs were taken. In subsequent use of the aerosol delivery device, following the N puffs, the controller may after each puff calculate a current rate of aerosol generation for the last Q puffs, where Q<N. For example, Q may comprise less than 50 puffs, less than 40 puffs, less than 30 puffs, less than 20 puffs, or less than 10 puffs. If the rate of aerosol generation for the last Q puffs (in terms of mass of aerosol per unit time) exceeds the rate of aerosol generation for the last N puffs (in terms of mass of aerosol per unit time), the controller 22 may establish a cessation control parameter comprising a lockout period for which aerosol cannot be generated by the device regardless of user demand. The length of the lockout period (e.g. in terms of seconds, minutes, or hours) may be proportional to the degree to which the rate for the last Q puffs exceeds the rate for the last N puffs. In a modification of this scheme, the cessation control parameter may be substituted for a power control parameter, such that power delivery to the heater is increased in dependence on a difference between a rate of aerosol delivery during the last Q puffs and the rate of aerosol delivery during the last N puffs.

In any of the embodiments described herein, the controller may optionally be configured to establish usage data based on monitoring the aspect of usage of the aerosol delivery system by the user over a plurality of puffs comprising between 20 and 150 puffs, wherein the controller is further configured to control an aspect of operation of the aerosol delivery system based on the established usage data, and wherein the selection of the usage data to use is based on a context in which the control the aspect of operation of the aerosol delivery system is to be implemented.

Thus, in some embodiments, the context comprises a day of the week, and the usage data comprises usage data based on monitoring the aspect of usage of the aerosol delivery system by the user over a plurality of puffs comprising between 20 and 150 puffs on a given day of the week, wherein the day of the week for which the usage data is derived is a preceding instance of the same day of the week as a day of the week in which the aspect of operation of the aerosol delivery system is to be controlled based on the usage data. Thus, during a Monday, the controller may control an aspect of operation of the aerosol delivery system as described herein on the basis of usage data, the usage data being established based on monitoring any aspect of usage of the aerosol delivery system by the user as described herein over a plurality of puffs comprising between 20 and 150 puffs, where the usage data is obtained by monitoring the aspect of usage during a previous Monday (i.e. the 20 to 150 puffs occurred between 0000H and 2400H on a previous Monday). It will be appreciated that the selection of Monday is not significant, and this principle may be applied in the same manner in respect of any other day of the week. This embodiment recognises that usage may in many cases be characteristically different on different days, and in particular, usage on weekend days may be characteristically different to usage on weekdays, due to a user having different usage patterns depending on whether they are on a working day (where use of their device may be restricted), or on a non-working day (where use of their device may be less restricted). Thus more generally, usage data for a weekend day (which could be either Saturday or Sunday) may be used to determine how to control an aspect of operation of the aerosol delivery system in a subsequent weekend day (which could also be either Saturday or Sunday), and/or usage data for a weekday (which could be any of Monday to Friday) may be used to determine how to control an aspect of operation of the aerosol delivery system in a subsequent weekday (which could also be any of Monday to Friday). Thus usage data relating to a Saturday may be used to control an aspect of operation of the device on a Sunday, or usage data relating to a Monday may be used to control an aspect of operation of the device on a Thursday, and so on.

More generally still, a context used to determine what usage data to use when controlling an aspect of operation of the aerosol delivery system may be based on selection of one from among a plurality of characteristic/distinct periods of time, between which types of periods usage of the device by a user may generally be different, but wherein usage of the device may be similar for different instances of the same type of period. For example, periods of time where the user is at work, or at home, or on vacation, may comprise characteristic/distinct types of time period, and the control of an aspect of operation of the aerosol delivery system in one of these characteristic/distinct time periods may be based on usage data established based on monitoring an aspect of usage of the aerosol delivery system by the user over a plurality of puffs comprising between 20 and 150 puffs where the 20 to 150 puffs occurred during a previous instance of the same type of characteristic/distinct time period. For example, when the user is determined to be on vacation (as determined, for example, based on GPS information indicating the user has travelled to a different country or city, or based on a user explicitly indicating they are on vacation via an input to an app running on a smartphone with a data connection to the aerosol delivery system, or based on an app running on the smartphone accessing an electronic calendar for the user and establishing the user is on vacation), an aspect of operation of the aerosol delivery system may be based on usage data established when the user was previously determined (via any of the previously mentioned approaches), to be on vacation. It will be appreciated that reference to vacation is merely one example of a context, and that this concept may be generalised to other distinct/characteristic periods of time (for example, ‘at work’, ‘at home’, ‘on sick leave’, or any other type of period known to the skilled person).

As described herein, in embodiments of the present disclosure, the context may be determined on the basis of a location of the aerosol delivery system as established on the basis of, for example, information about location of the aerosol delivery device, or an external electronic device such as a smartphone, based on GPS location tracking via a GPS tracking module located in the aerosol delivery system or smartphone, according to GPS tracking approaches known to the person skilled in the art. Thus, usage data may comprise usage data based on monitoring an aspect of usage of the aerosol delivery system by the user over a plurality of puffs comprising between 20 and 150 puffs, when the aerosol delivery system (or user smartphone) is in a particular location or region, and the aspect of operation of the aerosol delivery system is controlled based on the usage data when the aerosol delivery system (or user smartphone) is located in the same location or region. Thus if the aerosol delivery system or user smartphone is located in a particular building, street, city, district, or country, an aspect of operation of the aerosol delivery system may be based on usage data based on monitoring an aspect of usage of the aerosol delivery system by the user over a plurality of puffs comprising between 20 and 150 puffs, when the aerosol delivery device or user smartphone was located in the same building, street, city, district, or country.

Thus there has been described an aerosol delivery device, the aerosol delivery device comprising: a sensor configured to monitor an aspect of usage of the aerosol delivery system by a user; and a controller configured to establish usage data based on monitoring the aspect of usage of the aerosol delivery system by the user over a plurality of puffs comprising between 20 and 150 puffs; wherein the controller is further configured to control an aspect of operation of the aerosol delivery device based on the established usage data.

The various embodiments described herein are presented only to assist in understanding and teaching the claimed features. These embodiments are provided as a representative sample of embodiments only, and are not exhaustive and/or exclusive. It is to be understood that advantages, embodiments, examples, functions, features, structures, and/or other aspects described herein are not to be considered limitations on the scope of the invention as defined by the claims or limitations on equivalents to the claims, and that other embodiments may be utilised and modifications may be made without departing from the scope of the claimed invention. Various embodiments of the invention may suitably comprise, consist of, or consist essentially of, appropriate combinations of the disclosed elements, components, features, parts, steps, means, etc, other than those specifically described herein. In addition, this disclosure may include other inventions not presently claimed, but which may be claimed in future. The delivery system described herein can be implemented as a combustible aerosol provision system, a non-combustible aerosol provision system or an aerosol-free delivery system.

1. An aerosol delivery device, the aerosol delivery device comprising:
a sensor configured to monitor an aspect of usage of the aerosol delivery system by a user; and
a controller configured to establish usage data based on monitoring the aspect of usage of the aerosol delivery system by the user over a plurality of puffs comprising between 20 and 150 puffs; wherein
the controller is further configured to control an aspect of operation of the aerosol delivery device based on the established usage data.

2. (canceled)

3. The aerosol delivery device of claim 1, wherein the number of puffs in the plurality of puffs is determined based on a number of puffs taken by a user in at least one predefined period of time.

4. The aerosol delivery device of claim 3, wherein the predefined period of time has a duration of 24 hours.

5. The aerosol delivery device of claim 1, wherein the number of puffs in the plurality of puffs is determined based on establishing a characteristic periodicity in usage of the aerosol delivery system by the user, wherein the periodicity is established based on analysis of usage data.

6. The aerosol delivery device of claim 1, wherein the number of puffs in the plurality of puffs is determined based on establishing a number of puffs over which an average value of a parameter comprised in usage data is determined to be characteristic of an aspect of usage of the aerosol delivery system by a user.

7. (canceled)

8. The aerosol delivery device of claim 6, wherein the aspect of operation is controlled for a second plurality of puffs based on usage data associated with a first plurality of puffs, wherein the second plurality of puffs also comprises between 20 and 150 puffs.

9. The aerosol delivery device of claim 8, wherein the first and second plurality of puffs overlap.

10. (canceled)

11. (canceled)

12. The aerosol delivery device of claim 8, wherein the aspect of operation is controlled for a puff n of the second plurality of puffs based on usage data associated with a puff n of the first plurality of puffs.

13. The aerosol delivery device of claim 8, wherein the aspect of operation is controlled for a puff n of the second plurality of puffs based on usage data for a subset of puffs of the first plurality of puffs, the subset of puffs being centred on a puff n of the first plurality of puffs.

14. The aerosol delivery device of claim 1, wherein the aspect of operation is controlled by setting a power level to be supplied to an atomiser in a puff.

15. The aerosol delivery device of claim 1, wherein the aspect of operation is controlled by determining a rate of aerosol generation based on the usage data, and determining a rate of aerosol generation to be applied during one or more puffs based on the determined rate of aerosol generation.

16. The aerosol delivery device of claim 1, wherein the usage data comprises at least one of a puff timing, puff duration, puff strength, and a power delivered to an atomiser during each puff.

17. The aerosol delivery device of claim 1, wherein the usage data comprises information about the location of a user of the aerosol delivery device.

18. The aerosol delivery device of claim 1, wherein the usage data comprises information about one or more control parameters input by a user of the aerosol delivery system

19. The aerosol delivery device of claim 1, wherein the usage data comprises information about at least one of a temperature of a user's skin, a heart rate of a user, and an acceleration of the device.

20. (canceled)

21. The aerosol delivery device of claim 1, wherein the aerosol delivery system comprises transceiver circuitry configured to transmit usage data to an external electronic device over a data communication interface, and to receive from the external electronic device over the data communication interface at least one control parameter to control an aspect of operation of the aerosol delivery system, wherein the control parameter is derived based on the usage data.

22. (canceled)

23. The aerosol delivery device of claim 1, wherein the controller is configured to establish a current context for the aerosol delivery device, and to control the aspect of operation of the aerosol delivery system based on usage data established based on monitoring of the aspect of usage of the aerosol delivery system during a period of time associated with a previous instance in which the aerosol delivery device was established to be associated with the same context.

24. The aerosol delivery device of claim 23, wherein the context comprises a predefined period of time.

25-27. (canceled)

28. A method of controlling an aerosol delivery device, comprising a sensor and a controller, wherein the method comprises controlling the aerosol delivery system to:
monitor, using the sensor, an aspect of usage of the aerosol delivery system by a user;
establish, using the controller, usage data based on monitoring the aspect of usage of the aerosol delivery system by the user over a plurality of puffs comprising between 20 and 150 puffs;
control, using the controller, an aspect of operation of the aerosol delivery device based on the established usage data.

29. A computer readable storage medium comprising instructions which, when executed by a processor, performs the method of claim 28.
